(19) United States
(12) Patent Application Publication
(10) Pub. No.: US 2019/0255036 A1
(43) Pub. Date: Aug. 22, 2019

(54) COMPOUND AND METHOD FOR REDUCING NEUROPATHIC PAIN AND DEPRESSION

(71) Applicant: Alexander Kariman, Rockville, MD (US)

(72) Inventor: Alexander Kariman, Rockville, MD (US)

(21) Appl. No.: 16/182,078

(22) Filed: Nov. 6, 2018

Related U.S. Application Data
(63) Continuation-in-part of application No. 15/898,232, filed on Feb. 16, 2018.

(51) Int. Cl. A61K 31/475; A61K 45/06; A61K 36/74; A61K 31/381; A61K 31/485; A61K 31/437

(52) U.S. Cl. CPC A61K 31/475; A61K 45/06; A61K 31/437; A61K 31/381; A61K 31/485; A61K 36/74

(57) ABSTRACT

The disclosed invention generally relates to pharmaceutical and nutraceutical compounds and methods for reducing neuropathic pain and depression caused by toxicity, metabolism abnormality, trauma, compression, autoimmune abnormality, congenital/hereditary abnormality, infection, abnormally activated neuronal circuits, emotional stress, chemical imbalance, mental health disorder, or other causes in subjects in need thereof. The disclosed invention further relates to naturally occurring, synthetic and semi-synthetic multifunctional compounds that exhibit antidepressant and analgesic activity where said compound contains a pharmacologically inactive substance.

### FIELD OF THE INVENTION

The present invention relates to pharmaceutical and nutraceutical compounds and methods for reducing neuropathic pain and depression caused by toxicity, metabolism abnormality, trauma, compression, autoimmune abnormality, congenital/hereditary abnormality, infection, abnormally activated neuronal circuits, emotional stress, chemical imbalance, mental health disorder, or other causes.

### BACKGROUND OF THE INVENTION

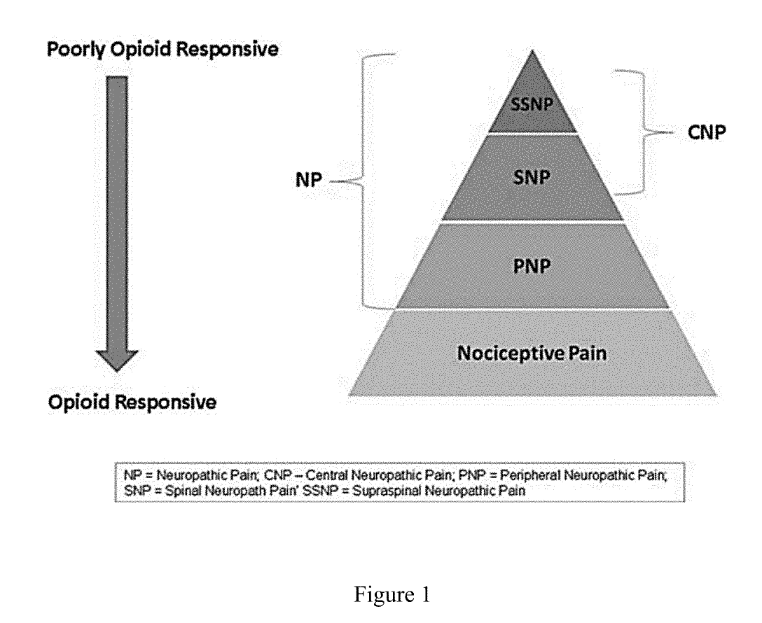

Neuropathic pain is often a chronic and disabling condition that is poorly responsive to usual analgesic drugs (Salvat, Yalcin, Muller, & Barrot, 2018). The treatment of neuropathic pain continues to be difficult and limited in options. Previously, neuropathic pain has also been reported to respond poorly to treatment with opioids, a phenomenon that is in contrast to the enhanced effect of opioid agonists in inflammatory pain (Amer & Meyerson, 1988) (Stein, 1995) (Ossipov, Kovelowski, & Porreca, 1995). However, recent clinical studies, indicate that neuropathic pain is not resistant to opioids, and patients with peripheral and central neuropathic pain may benefit from therapy with oral opioids such as methadone and oxycodone (FIG. 1) (Smith, 2012) (Watson & Babul, Efficacy of oxycodone in neuropathic pain: a randomized trial in postherpetic neuralgia., 1998) (Rowbotham, Twilling, Davies, & et al., 2003) (Zorn & Fudin, 2011) (Gagnon, Almahrezi, & Schreier, 2003).

Many studies have shown the effectiveness of mu-opioid receptor agonists and demonstrated that mu-opioid receptors contribute to the control of mechanical allodynia in neuropathic pain (Mansikka, Zhao, Sheth, & et al., 2004) (Finnerup, Sindrup, & Jensen, 2010). Indeed, mu-opioid receptor knockout mice showed an increase in mechanical allodynia under neuropathic pain induced by partial sciatic nerve ligation (Nadal, Baños, Kieffer, & Maldonado, 2006) (Gaveriaux-Ruff, Nozaki, & Nadal, 2011). Furthermore, mu-opioid receptor agonists attenuate mechanical allodynia in sciatic nerve-ligated mice (Mansikka, Zhao, Sheth, & et al., 2004). This suggests that the mu-opioid receptor contributes to alleviating mechanical allodynia (Matsumoto, Narita, Muramatsu, & et al., 2014).

There is, however, evidence that delta-opioid receptors contribute to reducing neuropathic pain when activated with mu-opioid receptors (Nadal, Banos, Kieffer, & Maldonado, 2006) (Gaveriaux-Ruff, Nozaki, & Nadal, 2011). Nevertheless, in other cases delta-opioid receptors did not demonstrate a significant impact on allodynia in animal models when tested directly. For instance, the results of one study suggest that the ventrolateral orbital cortex (VLO) is involved in opioid-induced anti-allodynia, and μ- but not δ- and κ-opioid receptor mediates these effects in the rat with neuropathic pain-microinjection of opioid receptor agonists into VLO had no effect on the allodynia (Zhao, Wang, Jia, & Tang, 2006).

After reviewing the available analgesics that may be beneficial for alleviating the intense perception of algesia, Smith (2012) concludes that evaluation and treatment of neuropathic pain is extremely challenging and complex: “[E]very individual patient is different, individual opioid analgesic agents are different, opioid receptors and their splice variants are different (and may produce different effects), G-protein subtypes are different and neuropathic pain states are different.”

Although, there is no robust evidence that any specific opioid agent is better than any other opioid at effectively treating neuropathic pain, it is conceivable that some opioids/opioid-like analgesic agents may be particularly well suited for alleviating neuropathic pain in certain patients suffering from such pain (Smith, 2012).

Neuropathic pain can be considered as a form of chronic stress that may share common neuropathological mechanism between pain and stress-related depression and respond to similar treatment. Therefore, neuropathic pain, and specifically allodynia, is commonly treated with antidepressants. There is extensive literature supporting the use of the antidepressants, such as amitriptyline, in neuropathic pain (Watson & Watt-Watson, 1999) (Raja, Haythornthwaite, Pappagallo, & et al., 2002) (Zorn & Fudin, 2011).

In fact, the first-line treatments presently recommended against neuropathic pain are either anticonvulsant drugs, such as gabapentinoids, or antidepressant drugs, such as tricyclic antidepressants (TCAs) or more selective serotonin and noradrenaline reuptake inhibitors (SSNRIs) (Salvat, Yalcin, Muller, & Barrot, 2018). In general, first-line therapy comprises tricyclic antidepressants, serotonin and norepinephrine reuptake inhibitors, calcium channels a2-ligands and topical lidocaine (Amorim, 2015). Second-line therapy includes strong opioids, such as tramadol (Amorim, 2015). Third-line therapy includes other antidepressants and anticonvulsants, N-methyl-D-aspartate (NMDA) receptor antagonists, topical capsaicin and gamma-aminobutyric acid (GABA) B receptor agonists (Amorim, 2015). Combination therapy is also recommended (Amorim, 2015).

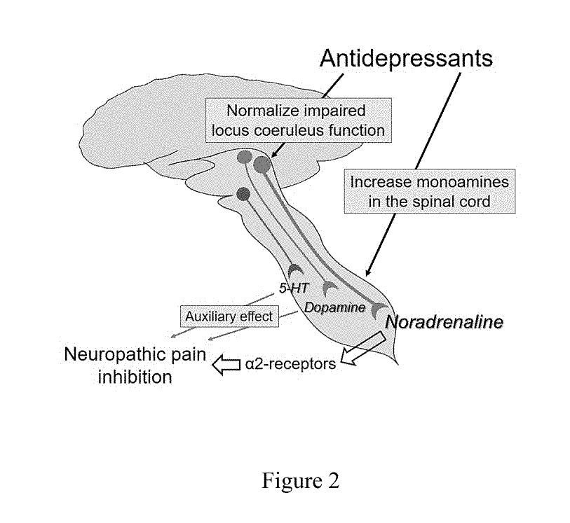

Why antidepressants are effective for treatment of neuropathic pain and the precise mechanisms underlying their effects, however, remain unclear. The inhibitory effects of antidepressants for neuropathic pain, such as tricyclic antidepressants and serotonin noradrenaline reuptake inhibitors, manifest more quickly than their antidepressant effects, suggesting different modes of action (Obata, 2017). Recent studies of animal models of neuropathic pain revealed that noradrenaline is extremely important for the inhibition of neuropathic pain (Obata, 2017). First, increasing noradrenaline in the spinal cord by reuptake inhibition directly inhibits neuropathic pain through a2-adrenergic receptors; second, increasing noradrenaline acts on the locus coeruleus and improves the function of an impaired descending noradrenergic inhibitory system (FIG. 2) (Obata, 2017). Serotonin and dopamine may reinforce the noradrenergic effects to inhibit neuropathic pain, according to Obata (2017).

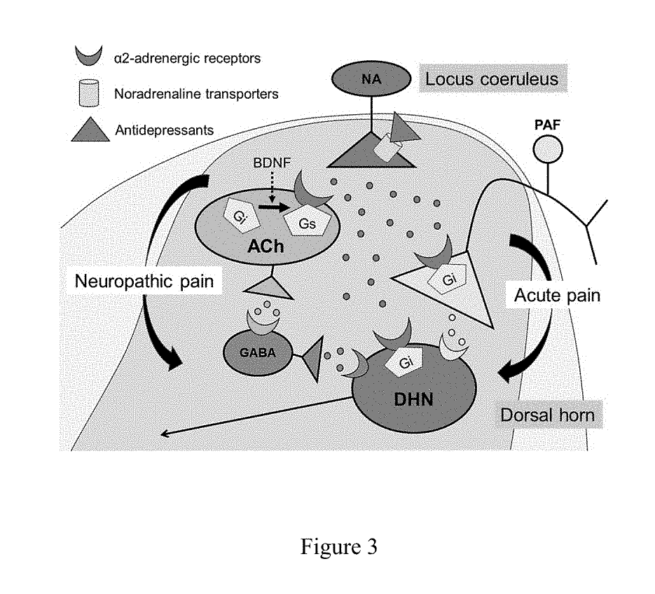

Antidepressants increase noradrenaline via blocking of noradrenaline transporters at the terminal of the descending noradrenergic fiber from the locus coeruleus (Obata, 2017). Noradrenaline inhibits acute pain through a2-adrenergic receptors by pre-synaptic (inhibit neurotransmitters release) and post-synaptic (hyperpolarize cell membranes) mechanisms (Obata, 2017). In neuropathic pain states, however, a2-adrenergic receptors in the cholinergic interneurons change from inhibitory to excitatory through G-protein switch (from Gi to Gs) by the effect of brain-derived neurotrophic factor (BDNF) increasing after nerve injury (Obata, 2017). Released acetylcholine bind to muscarinic receptors, by which produce analgesia thorough GABA release (FIG. 3) (Obata, 2017).

In one study, comparing the analgesic and cognitive effects of opioids with those of TCA and placebo in the treatment of postherpetic neuralgia (PHN), opioids and TCA reduced pain more than placebo with no appreciable effect on cognitive measure (Raja, Haythornthwaite, Pappagallo, & et al., 2002). The trend favoring opioids over TCA fell short of significance, however, according to Raja et al., (2002) and reduction in pain with opioids did not correlate with the TCA in this case. Nevertheless, treatment with opioids and TCA in this study resulted in greater pain relief compared with placebo (Raja, Haythornthwaite, Pappagallo, & et al., 2002).

Despite the addiction and other concerns, morphine and its derivatives remain to be the primary medicines to treat sever pain. Morphine is an analgesic indispensable for an improvement in quality of life (QOL) of a patient with severe pain. However, morphine has problems of, for example, having low bioavailability and causing various side effects, such as formation of analgesic resistance and physical or psychological dependence due to continued use, nausea and vomiting, constipation, sleepiness, and most importantly respiratory depression.

With that said, the advent of a potent and more safe analgesic, serving as a substitute for morphine, oxycodone, methadone, and the like, has long been needed. In search of such analgesics, investigations of synthetic analogs were performed with chemical modification of a morphine molecule, starting the 1920s and until the present day. Many compounds were synthesized since then and evaluated. However, there are not many examples of an opioid analgesic substance that is as effective as morphine or its synthetic analogs but safer than them. Current research has focused on an analgesic action of morphine and efforts are currently made to elucidate a molecular mechanism of analgesia on the basis of, for example, classification of opioid receptors (δ-, μ-, and κ-receptors) and determination of amino acid sequences thereof. However, there are complicated interactions among these receptors, and a logical methodology for separating an analgesic property from side effects, such as a narcotic property, has not been established to date.

There are other opioid pharmaceutical products exist which provide analgesic relief but at the same time may have fewer side effects and lesser addiction liability. For example, buprenorphine, sold under the brand name Subutex, among others, is an opioid used to treat opioid addiction, acute pain, and chronic pain. It comes in injectable, transdermal, sublingual, and other dosage forms. Maximum pain relief is generally achieved within an hour with effects lasting up to 24 hours. Buprenorphine affects different types of opioid receptors in different ways. In simplified terms, buprenorphine can essentially be thought of as a non-selective, mixed agonist-antagonist opioid receptor modulator, acting as a weak partial agonist of the MOR, an antagonist of the KOR, an antagonist of the DOR, and a relatively low-affinity, very weak partial agonist of the ORL-1. Full analgesic efficacy of buprenorphine requires both exon 11 and exon 1-associated μ-opioid receptor splice variants. Side effects may include respiratory depression, sleepiness, adrenal insufficiency, QT prolongation, low blood pressure, allergic reactions, and opioid addiction. Among those with a history of seizures, there is a risk of further seizures. Opioid withdrawal following stopping is generally mild.

Tramadol is another opioid pharmaceutical product that provides analgesic relief but has fewer side effects and reduced addiction liability. Tramadol is sold under the brand name Ultram, among others, and it is an opioid pain medication used to treat moderate to moderately severe pain. When taken by mouth in an immediate-release formulation, the onset of pain relief usually occurs within an hour. It is often combined with paracetamol (acetaminophen) as this is known to improve the efficacy of tramadol in relieving pain. It works by binding to the μ-opioid receptor and as a serotonin-norepinephrine reuptake inhibitor (SNRI). Tramadol is in the benzenoid class, and in the body, it is converted to desmetramadol, which is a more potent opioid. Common side effects include: constipation, itchiness and nausea. Serious side effects may include seizures, increased risk of serotonin syndrome, decreased alertness, and drug addiction. Long-term use of high doses of tramadol will cause physical dependence and withdrawal syndrome. Tramadol withdrawal typically lasts longer than that of codeine and other weak opioids (seven days or more of acute withdrawal symptoms). However, according to a 2014 report by the World Health Organizations Expert Committee on Drug Dependence, evidence of tramadol physical dependence was considered minimal. Consequently, tramadol is generally considered to be a drug with low potential for dependence.

There are a number of opioid receptor-modulating investigational analgesics that are currently under development for clinical use. For example: Axelopran/oxycodone-combination of a centrally active μ-opioid receptor agonist and a peripherally selective μ-, κ-, and δ-opioid receptor antagonist; Cebranopadol (GRT-6005)-non-selective μ-opioid receptor, nociceptin receptor, and δ-opioid receptor full agonist and κ-opioid receptor partial agonist; Desmetramadol (O-desmethyltramadol; Omnitram)-μ-opioid receptor agonist, norepinephrine reuptake inhibitor (NRI), and 5-HT2C receptor antagonist; Difelikefalin (CR845, FE-202845)-peripherally selective κ-opioid receptor agonist. Lexanopadol (GRT-6006, GRT13106G)-non-selective opioid receptor agonist; Nalbuphine sebacate (dinaphine, sebacoyl dinalbuphine ester; LT-1001)-long-lasting prodrug of nalbuphine, μ- and κ-opioid receptor partial agonist; NKTR-181-selective μ-opioid receptor full agonist that slowly enters the brain; Oliceridine (TRV130)-μ-opioid receptor biased agonist; Oxycodone/naltrexone-combination of a μ-opioid receptor agonist and a μ- and κ-opioid receptor antagonist, and others.

As already stated, one objective of the present invention is to provide compounds that have potent analgesic and antidepressant actions, which may offer an alternative to currently used opioid analgesics and antidepressants. The novel compositions and treatment methods, with several variations, some of which are exemplified herein, exhibit a potent analgesic and antidepressant actions, featuring good pharmacokinetic potential for an orally administered medication. The present invention has relied on multiple new scientific findings, experiments and anecdotal evidence obtained through this research. The inventor believes that this invention is unique and different from the existing art related to Mitragyna speciosa-based compounds, processes, and methods. Some of the existing art is briefly outlined below.

The U.S. Pat. Application No. 15,032,070, referenced herein, discloses a formula of compound that has an analgesic effect and high metabolic stability. The invention further provides the following: an analgesic obtained from the compound, a salt thereof, or solvates of the compound and salt; a pharmaceutical composition containing the compound, a salt thereof, or solvates of the compound and salt; an analgesic treatment method using the compound, a salt thereof, or solvates of the compound and salt; and a use of the compound, a salt thereof, or solvates of the compound and salt, in the production of an analgesic composition.

The U.S. Pat. No. 13,024,298, referenced herein, discloses hybrid opioid compounds, mixed opioid salts, compositions comprising the hybrid opioid compounds and mixed opioid salts, and methods of use thereof. More particularly, in one aspect the hybrid opioid compound includes at least two opioid compounds that are covalently bonded to a linker moiety. In another aspect, the hybrid opioid compound relates to mixed opioid salts comprising at least two different opioid compounds or an opioid compound and a different active agent. Also disclosed are pharmaceutical compositions, as well as methods of treating pain in humans using the hybrid compounds and mixed opioid salts.

The U.S. Pat. No. 13,024,298, referenced herein, discloses a compound that is a pharmaceutically acceptable salt or ester thereof, and a method of treating a subject afflicted with pain, a depressive disorder, a mood disorder or an anxiety disorder by administering the compound to the subject.

### SUMMARY OF THE INVENTION

The following description presents a simplified view of one or more aspects of the proposed invention. This summary is not an extensive overview of all the contemplated embodiments and implementations. It is intended to neither identify key or critical elements of all features, nor delineate the scope of any or all facets. Its sole purpose is to present some concepts of one or more aspects in a simplified form.

The inventors hereby propose that it is conceivable that an opiate analgesic, having also antidepressant properties, would be a superior neuropathic pain treatment option. Also, an opioid analgesic having antidepressant actions may be superior for threatening a depression. Especially, if such opioid analgesic has a reduced side effects profile compare to the opioid medications used today. Peculiarly, but according to Raja et al., (2002) more patients in a study comparing opioid, TCA, and placebo treatments of postherpetic neuralgia (PHN) preferred opioids than TCA. Opioids effectively treated PHN without cognition impairment (Raja, Haythornthwaite, Pappagallo, & et al., 2002).

After conducting an extensive research, the inventors have developed a hypothesis, elucidating the proposed novel mechanism of action, and subsequently developing a set of requirements that a novel pharmaceutical shall meet in order to demonstrate superior analgesic and antidepressant qualities. Next, the inventors have identified a number of substances, where several of them are naturally occurring, that meet the desired requirements, demonstrating essentially the expected in vivo and in vitro results.

These substances have been known to potentially exhibit antinociceptive qualities in animal models, but their application as multi-functional antidepressant/neuro-analgesics for peroral administration, and the related pharmaceutical formulations, as well as treatment protocols were not previously known. Nor the pharmacodynamic parameters related to the antidepressant/neuro-analgesic activity for peroral and other administration routes were elucidated; likewise, their pharmacokinetic properties and patient dosing relevant to the antidepressant/neuro-analgesic activity.

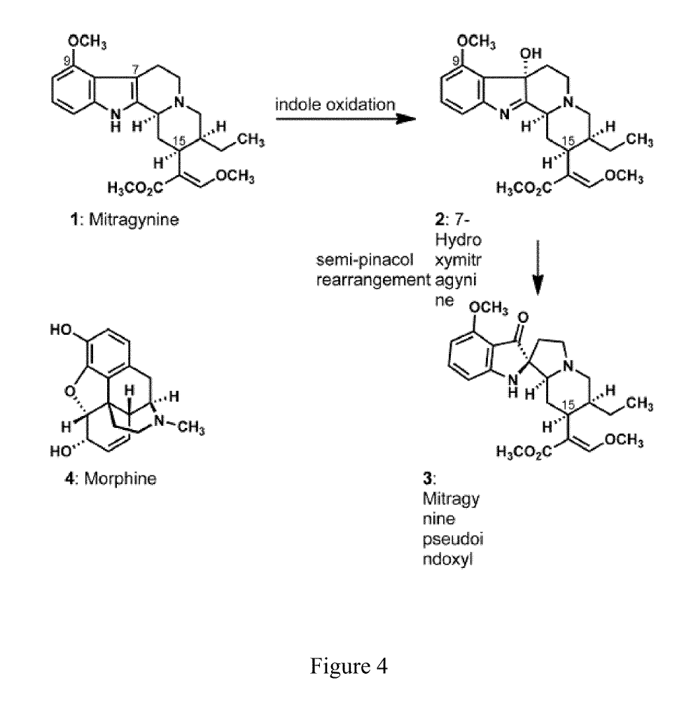

The substances identified are 7-hydroxymitragynine, mitragynine and mitragynine pseudoindoxyl (FIG. 4). These compounds were not previously used for neuropathic pain management, and as inventors have discovered, they can be a potential breakthrough in treatment of neuropathic pain, such as allodynia in the Complex Regional Pain Syndrome (CRPS), for the following reasons:
1. They exhibit opiate analgesia that involves mu-opioid receptors with poetically reduced side effects.
2. They exhibit antidepressant effects mediated by an interaction with serotonergic system, stimulating α2-adrenoceptors and/or blocking 5-HT2A receptors, as well as stimulating mu-opioid receptors (MOR) (partial agonism) and blocking kappa-opioid receptors (KOR) (antagonism)—the activities that have been shown to elicit antidepressant effects in animals and humans.

The reduced opiate analgesic side effects, as mentioned above, can be attributed to the following aspects, where 7-hydroxymitragynine, mitragynine and mitragynine pseudoindoxyl are:
1. Agonists at human mu-opioid receptors (hMOR) and competitive antagonists at human delta-opioid receptors (hDOR).
2. G-protein-biased agonists of the mu-opioid receptors, which do not recruit β-arrestin.

Concerning the first aspect, there is evidence supporting a modulatory role for the delta-opioid receptor in morphine analgesia, tolerance, and physical dependence. Morphine tolerance has been observed to produce changes in delta-opioid receptor binding capacity in mice and rats (Holaday, Hitzemann, Curell, & et al., 1982) (Rothman, Danks, Jacobson, & et al., 1986), whereas selective delta receptor blockade prevents the development of morphine tolerance and dependence in mice (Abdelhamid, Sultana, Portoghese, & Takemori, 1991) (Miyamoto, Portoghese, & Takemori, 1993). Physiological and biochemical evidence indicating a close association of delta and mu binding sites (perhaps in an opioid receptor complex) has been also reported by Traynor et al., (1993). It may account for the interaction between delta receptor ligands and morphine (Kest, Lee, McLemore, & Inturrisi, 1996).

Abdelhamid et al., (1991) reports that the use of the delta-opioid receptor antagonists provides a way to prevent opiate tolerance and physical dependence without compromising the antinociceptive activity of mu opioid receptor agonists such as DAMGO and morphine. Hepburn et al., (1997) suggest that delta antagonists may have potential clinical application for decreasing the rapid development of tolerance to opiate-induced analgesia, while allowing for the development of protective tolerance to respiratory depression (Zhu, King, Schuller, & et al., 1999). Zhu et al. (1999) have also demonstrated a loss of morphine tolerance in delta opioid receptor knockout mice.

In addition, Suzuki et al., (1997) reported that pretreatment with not only delta 2 but also delta 1 opioid receptor antagonist during chronic morphine treatment reduced naloxone-precipitated withdrawal dependence on morphine. These findings suggest that both delta 2 and delta 1 opioid receptors may play an essential role in modulating the development of physical dependence.

Therefore, inventors believe that this agonist activity at hMOR and competitive antagonist activity at hDOR is partially responsible for the reduced side effects profile of 7-hydroxymitragynine, mitragynine and mitragynine pseudoindoxyl.

Concerning the second condition, it is possible that functionally selective opioid receptor agonists, which preferentially activate only certain downstream signaling pathways, produce lower side effects. In particular, some evidence indicates that MOR agonists biased toward G-protein signaling over β-arrestin signaling display less respiratory depression, tolerance development, and constipation, while remaining potent analgesics (Groer, Tidgewell, Moyer, & et al., 2007) (Lamb, Tidgewell, Simpson, & et al., 2012) (DeWire, Yamashita, Rominger, & et al., 2013) (Soergel, Subach, Burnham, & et al., 2014).

Both the analgesic and respiratory suppressive effects of opioids are due to the activation of mu-opioid receptors (Dahan, Sarton, Teppema, & et al., 2001) (Matthes, Maldonado, Simonin, & et al., 1996). As a G-protein-coupled receptor (GPCR), MOR also interacts with b-arrestins, scaffolding proteins that serve to regulate or facilitate subsequent GPCR signaling. In studies spanning more than a decade, researchers have shown that the interaction between MOR and b-arrestin 2 may drive many of the unwanted side effects of MOR activation (Bohn, Lefkowitz, Gainetdinov, & et al., 1999) (Bohn, Gainetdinov, Lin, & et al., 2000) (Bu, Liu, Tian, & et al., 2015) (Li, Liu, Liu, & et al., 2009) (Raehal, Schmid, Groer, & Bohn, 2011).

B-arrestin 2-knockout mice, for example, display enhanced and prolonged morphine-induced antinociception yet are protected from morphine-induced respiratory suppression (Bohn, Lefkowitz, Gainetdinov, & et al., 1999). These findings suggest that activating the MOR without engaging b-arrestin 2 regulation may be critically important for developing safer opioid analgesics (Schmid, Kennedy, Ross, & et al., 2017). Schmid et al., (2017) further asserts that β-arrestin-biased compounds, such as fentanyl, are more likely to induce respiratory suppression at weak analgesic doses, while G-protein signaling bias broadens the therapeutic window, allowing for antinociception in the absence of respiratory suppression.

There are a number of novel pharmaceuticals under development that utilize this principle in the hopes of reduced side effects. For instance, Oliceridine (developmental code name TRV-130; tentative brand name Olinvo)—an opioid drug that is under evaluation in the stage three human clinical trials for the intravenous treatment of severe acute pain (Trevena, Inc., 2018). Another drug, TRV-734, is similar to TRV-130 but for oral administration (Trevena, Inc., 2018). Both drugs are μ-opioid receptor biased agonists developed by Trevena. The developer claims that in in vitro research, Oliceridine elicits robust G-protein signaling, with potency and efficacy similar to that of morphine, but with less β-arrestin 2 recruitment and receptor internalization—thus, it may have fewer adverse effects than morphine.

It was shown that mitragynine, mitragynine pseudoindoxyl and 7-hydroxymitragynine are biased hMOR agonists because they activate GPCR but not β-arrestin. Silencing the β-arrestin pathway may decrease undesirable side effects, including respiratory depression, constipation, and tolerance, therefore a lower side effect profile can be achieved—in contrast, for example, with morphine and β-endorphin that are balanced MOR agonists which activate both GPCR and β-arrestin pathways.

These results show that mitragynine, mitragynine pseudoindoxyl and 7-hydroxymitragynine may have reduced side effect profile due to (a) being G-protein-biased agonists of the mu-opioid receptor that do not recruit β-arrestin following receptor activation; as well as (b) being hMOR agonists and competitive antagonists at hDOR. Therefore, the inventors propose that 7-hydroxymitragynine, mitragynine and mitragynine pseudoindoxyl may represent new opioid analgesics with further reduced side effects due to this dual action.

A number of studies have demonstrated that mitragynine exerts an antidepressant effect in animal behavioral models of depression (Abushwereb, Abdulatif, & Abdulmajeed, 2018) (Kruegel, Gassaway, & Kapoor, 2016) (Idayu, Hidayat, Moklas, & et al., 2011) (Kumarnsit, Vongvatcharanon, Keawpradub, & Intasaro, 2007) (Kumarnsit, Keawpradub, & Nuankaew, Effect of Mitragyna speciosa aqueous extract on ethanol withdrawal symptoms in mice., 2007).

It is proposed that mitragynine acts as both a low efficacy MOR partial agonist, and a KOR antagonist—both mechanisms have been shown to elicit antidepressant effects in animals (KOR antagonist: (Kruegel, Gassaway, & Kapoor, 2016) (Carr, Bangasser, Bethea, & et al., 2009), MOR agonist: (Gassaway, Rives, Kruegel, Javitch, & Sames, 2014)) and humans: For instance, ALKS 5461 (Buprenorphine/samidorphan) and CERC-501(LY-2456302) are prospective pharmaceuticals that utilize said KOR antagonism principle, which entered Phase-III and Phase-II clinical trials, respectively (Li, Sun, Chen, Yang, & et al., 2016). And an atypical antidepressant tianeptine (brand names Stablon and Coaxil among others (not FDA approved))—MOR agonist (with clinically negligible effects on the δ- and κ-opioid receptors) (Gassaway, Rives, Kruegel, Javitch, & Sames, 2014)). KOR antagonists oppose a variety of stress-related responses that are believed to be mediated through the endogenous KOR agonists, dynorphins (Kruegel, Gassaway, & Kapoor, 2016). Also, the apparent lack of abuse potential compare to MOR agonists makes them a more favorable treatment option.

The second proposed antidepressant mechanism of the claimed substances is the ability of mitragynine (as well as mitragynine pseudoindoxyl and 7-hydroxymitragynine, according to the inventors) to stimulate a2-adrenoceptors and/or block 5-HT2A receptors of serotonergic system (Abushwereb, Abdulatif, & Abdulmajeed, 2018).

Given the limited therapeutic options that are available for the management of neuropathic pain, and specifically allodynia, the findings proposed herein are increasingly relevant, providing primary data of the potential usefulness of 7-hydroxymitragynine, mitragynine pseudoindoxyl and 7-hydroxymitragynine in treating neuropathic pain. 7-Hydroxymitragynine, mitragynine pseudoindoxyl and mitragynine not only could induce a potent antinociceptive effect against acute pain, but they can potentially produce a complex analgesic effect against neuropathic pain via multiple mechanisms of action, benefiting a larger group of patients than the current medications.

As already explained, being a G-protein-biased hMOR agonist not recruiting (3-arrestin, as well as an hDOR competitive antagonist, 7-hydroxymitragynine, mitragynine pseudoindoxyl and mitragynine may have reduced side effects when compared with other opiates used for treatment of neuropathic pain.

In one embodiment, the proposed invention includes a gelatin capsule with a Mitragyna speciosa plant extract that contains 10 mg of 7-hydroxymitragynine dissolved in a pharmaceutically-acceptable medium chain triglyceride oil, and the extract further contains at least a trace amount of mitragynine, as well as at least a trace amount of one other of plurality of indole or oxindole alkaloids, which are co-extracted with 7-hydroxymitragynine from the plant material. Their respective ranges may vary according to the starting plant material and the extraction methodology used. The capsule further contains methyl- and propyl-parabens. The 7-hydroxymitragynine-containing plant extracts may be obtained by various processes of extraction from the plant material. The term “plant extract” is taken herein to refer to one or more plant extracts from any 7-hydroxymitragynine and/or mitragynine-containing plant; it can also be understood as synthesized 7-hydroxymitragynine, mitragynine and/or mitragynine pseudoindoxyl.

In another embodiment, the proposed invention includes a synthesis of mitragynine using one of the available techniques, such as the total synthesis proposed by Ma, et al., (2007) using an enantiospecific method for the synthesis of mitragynine via a regiospecific Larock heteroannulation, the asymmetric Pictet-Spengler reaction, and a Ni(COD)2 mediated cyclization serving as key steps. In another embodiment, a preservative-free injection solution containing 18 mg/mL of morphine sulphate and 7 mg/mL of mitragynine dissolved in a pharmaceutically-acceptable nanoemulsion carrier, having average droplet size in between 100-500 nm. The solution is packaged in a 20 mL ampule dose form.

In another embodiment, a morphine sulphate/mitragynine (10:1) injection is administered for intractable neuropathic pain using an intrathecal morphine pump in a patient with acute transverse myelitis. After 14 days of administration, the development of analgesia tolerance (analgesic resistance) was reduced by at least 30% in comparison with the morphine sulphate treatment alone. The patient was suffering from intractable neuropathic pain following acute transverse myelitis that was not relieved by combinations of nonsteroidal anti-inflammatory, anti-epileptic, antidepressant medications, or by acupuncture. Implantation of an intrathecal morphine/mitragynine pump would control the pain with less amount of morphine due to a solver development analgesic resistance.

In one embodiment, 7-hydroxymitragynine is obtained semi-synthetically as a result of oxidation of naturally derived mitragynine from Mitragyna speciosa plant. In one embodiment, the proposed invention includes an enteric-coated delayed release hard-pressed tablet that contains 5 mg of 7-hydroxymitragynine dispersed in 50 mg of ascorbic acid and Tween 80 surfactant. The active substance is released into the small intestine when taken with food, having approximately 6 hours delay. One tablet is administered to a subject in 6 hours for treatment of injury-related allodynia pain.

With peroral administration, 7-hydroxymitragynine slightly degrades to mitragynine in gastro-intestinal tract, and then significantly degrades (45%) to mitragynine as part of the metabolic conversion (Manda, Avula, & Ali, 2014). Therefore, both 7-hydroxymitragynine and mitragynine become bioavailable with peroral administration of 7-hydroxymitragynine, where the smaller quantities of mitragynine, being bioavailable, are therapeutically meaningful. Otherwise, absorption of peroral mitragynine is relatively slow, prolonged and incomplete, with a calculated absolute oral bioavailability value of 3.03%, measurable in plasma from 0.3 to up to 24 h after administration of the dose (Suhanya, Ramanathan, Ismail, & et. al., 2010).

In summary, the proposed invention provides methods and compounds for treatment of multiple diseases and disorders at various stages, and different patients potentially presenting different symptoms, and as such may require larger or smaller doses to achieve the desired efficacy. Besides, the different ratios of the Mitragyna speciosa plant alkaloids, other ingredients are required to achieve the desired effect, such as proper absorption.

In one aspect of the invention, titration of doses is beneficial to patients as they can take smaller doses of the medication to achieve efficacy. It is understandable that not all patients will require the same dose of medication, for example, patients of a larger build or faster metabolism may require a higher dose than that required by a patient that is of a smaller build or slower metabolism. In one embodiment said titration is adjusted with a time-release and point of release-tailored dosage forms. For instance, a soft-gelatin capsule designed to release medication in doses in certain parts of the digestive system to achieve the desired efficacy.

In another embodiment, the dose of medicament to be administered to a subject suffering from chronic pain is formulated such that a specific patient can titrate such dose and not develop significant tolerance to the medication; where the term “titrate” means that the patient is provided with a medication that is in such a form or engineered in such a way that smaller doses than the unit dose can be taken. In one embodiment, the titratable dosage forms are gel, gel spray, transdermal patch, liquid, vapor, and the like.

The unit dosage—defined as a maximum dose of medication that can be taken at any one time or within a specified dosage period—may range, in one embodiment, between 5 mg and 600 mg of said medicine for a patient that just starts using the medication, or, depending on the administration route and aforesaid variables, the dosage may fluctuate significantly, such that unit dosage may consist of multiple doses taken several times a day, especially for long-term use patients that have developed tolerance. Administration of the compound may be carried out by any of several suitable known means, including but not limited to intraperitoneal, subcutaneous, oral, intramuscular, intravenous, and other administration forms.

These and other embodiments and objects of the invention will become apparent upon further review of the specification and claims presented herein. Thus, the above and the following expressed embodiments and objects of the invention are not intended by the inventors to limit the scope of the invention.

### BRIEF DESCRIPTION OF THE DRAWINGS

The accompanying drawings, which are incorporated in and constitute a part of this specification, illustrate aspects of the present teachings and together with the description, serve to explain principles of the present teachings.

The FIG. 1, incorporated herein by reference, shows the relative overall opioid responsiveness for different categories of pain (Smith, 2012).

The FIG. 2, incorporated herein by reference, demonstrates inhibition of neuropathic pain by antidepressants (Obata, 2017).

The FIG. 3, incorporated herein by reference, provides a schematic illustration of analgesic effects of antidepressants and noradrenaline in the dorsal horn of the spinal cord (Obata, 2017). (Primary afferent fibers (PAF), noradrenaline (NA), dorsal horn neurons (DHN), acetylcholine (Ach), noradrenaline (red circle), acetylcholine (blue circle), GABA (green circle), excitatory neurotransmitters (pink circle).)

The FIG. 4, incorporated herein by reference, shows molecular structures of mitragynine (1), 7-hydroxymitragynine (2), mitragynine pseudoindoxyl (3), and morphine (4).

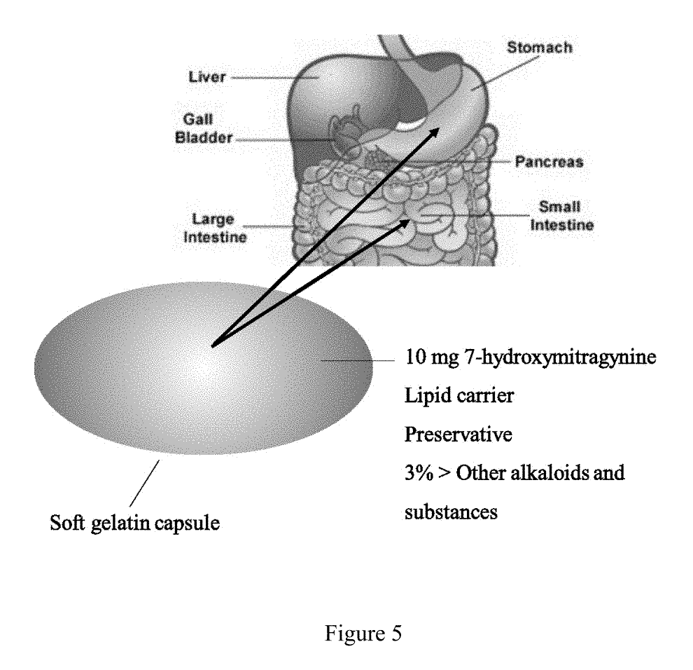

The FIG. 5, incorporated herein by reference, shows a variable-release soft-gelatin capsule pill, one of many possible dosage forms, that consists of predominantly type A or B gelatin, water, sorbitol, and encapsulates a compound containing a liquid mixture that includes 10 mg of 7-hydroxymitragynine, a lipid carrier, preservative, and less than 3% of other substances.

### DESCRIPTION OF EMBODIMENTS

Reference will now be made to embodiments, examples of which are illustrated in the accompanying material. In the following description, some details are set forth in order to provide understanding of the proposed invention. However, it will be apparent to one of ordinary skill in the art that the present invention may be practiced without these details. In other instances, well-known methods, procedures and components have not been described in detail so as not to unnecessarily obscure aspects of the embodiments.

It will also be understood that, although the terms first, second, etc., may be used herein to describe various elements, these elements should not be limited by these terms. These terms are only used to distinguish one element from another. For example, a first contact could be termed a second contact, and, similarly, a second contact could be termed a first contact, without departing from the scope of the present invention. The first contact and the second contact are both contacts, but they are not the same contact.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. Moreover, the term “or” is intended to mean an inclusive “or” rather than an exclusive “or.” That is, unless specified otherwise, or clear from the context, the phrase “X employs A or B” is intended to mean any of the natural inclusive permutations. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if is detected” may be construed to mean “upon determining” or “in response to determining” or “upon detecting (the stated condition or event)” or “in response to detecting (the stated condition or event),” depending on the context.

As used herein, the terms “related”, “in connection”, or “associated”, or “relevant”, and similar, depending on the context, means any association, whether direct or indirect, by any applicable criteria as the case may be.

The word “exemplary” is used herein to mean “serving as an example, instance, or illustration”. And no aspect of this disclosure shall be construed as preferred or advantageous over other aspects or designs unless expressly stated.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali metal salts, sodium, potassium or lithium.

As used herein, “treating” means preventing, slowing, halting, or reversing the progression of a disease or infection. Treating may also mean improving or attempting to improve one or more symptoms of a disease or infection.

As used herein, “trace amount” means as practiced in analytical chemistry—average concentration is less than 100 parts per million (ppm) measured in atomic count or less than 100 micrograms per gram.

As used herein, the percent by mass of a mixture is obtained by dividing the mass of each component by the total mass and multiply by 100 (Percent by mass=mass of component/total mass×100%). For example. a mixture that contains 1.203 g CaCO3 and 1.797 g NaCl is equal to CaCO3=40.10% and NaCl=59.90%.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The present invention provides a number of pharmaceutical compounds that represent a formulation of naturally occurring and/or synthetic alkaloids or their analogs (for the purpose of this document, may be used interchangeably) or other types of substances. An analog herein refers to a compound that is derived by chemical, biological or synthetic transformation of the naturally occurring substances, such as alkaloids of a Mitragyna speciosa plant, or synthetically obtained similar compounds.

The natural alkaloid compounds are readily obtained from plant tissue by suspending the tissue in an appropriate solvent to extract alkaloid compounds and other tissue components. Analytical purification of such an extract provides pharmaceutical grade alkaloid compounds. In one instance, mitragynine was found to be a pure compound upon spectroscopic analysis (including 1H and 13C NMR, IR, and mass spectrometry), and identified to be mitragynine by comparing the obtained spectra data with the published data (Shellard, Houghton, & Resha, 1978) (Houghton, Latiff, & Said, 1991).

Chittrakarn, et al. (2010), had performed an extraction and isolation of Mitragyna speciosa plant leaves red vein type. They were collected from Satoon province, in the southern part of Thailand during the months of February-May 2005. Air-dried leaves were pulverized by grinding and then macerated, at room temperature, with absolute methanol for 7 days, twice, while stirring 2-3 times/day. The extracts were mixed, filtered and concentrated using a rotary evaporator (BUCHI, B 169 Vacumn-System, Switzerland). Then they were freeze-dried (Corrosion Resistant Freezer Drier, FTS System, Inc., USA). The yield was 7.92% (w/w). (Chittrakarn, Keawpradub, Sawangjaroen, Kansenalak, & Benjamas, 2010)

According to Chittrakarn, et al., (2010), said isolation of dried product from the methanolic extract of Mitragyna speciosa plant leaves was dissolved in 10% acetic acid solution. This solution was shaken and left overnight. The acidic filtrate was washed with petroleum ether, adjusted to pH 9 with 25% ammonia solution, and then extracted with chloroform. The chloroform extract was washed with distilled water, dried over anhydrous sodium sulfate and evaporated to yield a dry crude alkaloid extract. According to the isolation procedure, Chittrakarn, et al., (2010) report that the yield of crude alkaloid extract was approximately 0.25% based on fresh weight of Mitragyna speciosa. An aliquot (2.5 g) was then subjected to silica gel column chromatography, eluted with 5% methanol in chloroform to obtain a major alkaloid (1.25 g), which appeared as a single spot on TLC analysis (four different solvent systems). Over all, the yield of mitragynine in the methanolic extract was approximately 1.56%. (Chittrakarn, Keawpradub, Sawangjaroen, Kansenalak, & Benjamas, 2010)

Alternatively, Mitragyna speciosa plant substances can be extracted from the plant tissue under supercritical conditions. Solvents used for supercritical extraction of alkaloids and other substances include without limitation: carbon dioxide, or other gases in isolation or combination with or without solvent modifiers, selected from ethanol, propanol, butanol, hexane, chloroform, dichloromethane, acetone, or any organic solvent capable of extracting such substances, and alcohol-water mixtures, for instance, water-ethanol or water-butanol mixtures, and others.

The present invention, in one embodiment, involves producing an extract from Mitragyna speciosa plant matter, containing mitragynine and 7-hydroxymitragynine. In one embodiment, the dried plant matter is ground and subjected to a CO2 extraction and the primary extract obtained is separated. Specifically, ground Mitragyna speciosa plant material is compressed and charged into an extraction vessel. CO2 is then introduced, having been brought to a temperature, in one embodiment, of approximately 60° C. and to a pressure of approximately 250 bars. When the CO2 enters into contact with the material to be extracted, it extracts the desired components, in particular comprising mitragynine and 7-hydroxymitragynine, as well as other indole or oxindole alkaloids. In one embodiment, the extraction method permits extracting various isomers of alkaloids, selectively obtained from industrial Mitragyna speciosa plant, also separating undesirable, alkaloids, waxes and removing the solvent.

The alkaloids, including mitragynine and 7-hydroxymitragynine, can be isolated from Mitragyna speciosa plant using the methanol extraction or any other suitable extraction method, or can be made synthetically as previously discussed. It is preferable, in one embodiment, that the extraction/production method yields substantially the mitragynine and 7-hydroxymitragynine that are believed to be the most effective alkaloids for pain management. There are also various other techniques that are known for extracting and isolating mitragynine and 7-hydroxymitragynine from other compounds in Mitragyna speciosa plant. For example, Pat. No. CN102048857B, describes a method for extracting alkaloids from Kratom. (CN Patent No. 102048857B, 2009)

The chemical total syntheses reported for several Mitragyna speciosa plant alkaloids are too complex to be used for economic production of these compounds. However, mitragynine can serve as a chemical precursor to the more potent 7-hydroxymitragynine, and the latter can serve as a starting point for mitragynine pseudoindoxyl.

It is not the purpose of present disclosure to provide particulars concerning the attainment of a colloidal formulation that is stable under a range of conditions. Though, in one embodiment, the disclosed compound with initial purity (HPLC) of mitragynine, mitragynine pseudoindoxyl and/or 7-hydroxymitragynine being at least 98% by area can achieve stability such that at least 95% by area remains in undegraded form after exposure of the compound to the storage conditions for twelve months, where the ambient temperature is between 20° C. and 40° C. and relative humidity is between 55% and 75%.

In one embodiment, the stability of said compound is attained by contacting a solution containing mitragynine, mitragynine pseudoindoxyl and/or 7-hydroxymitragynine into a solvent such as organic solvents, including acetone, acetic acid, alcohols, chloroform, diethyl ether solvents, and other solvents that can be used to dissolve the mitragynine, mitragynine pseudoindoxyl and/or 7-hydroxymitragynine; and in another embodiment, with addition of pharmaceutically acceptable buffers, stabilizers, and other pharmacologically inactive substances.

In one embodiment, the invented compound is in the form of micelles or liposomes that encapsulate mitragynine, mitragynine pseudoindoxyl and/or 7-hydroxymitragynine, and/or other alkaloids within the membrane of the micelles or liposomes. Within the context of the present technology, the term “micelle” refers to an aggregate of surfactant molecules dispersed in a liquid colloid, while “liposome” refers to a vesicle composed of a mono or bilayer lipid.

In yet another embodiment, other drugs, and pharmaceutically acceptable carriers, if present, may be in the lipophilic membrane or entrapped in the aqueous fluid that forms the core of the liposome. The entrapped alkaloids contribute to the stability of the micelle/liposome membranes, such that the micelle/liposomes formulations may be used as an improved, fast, reliable and efficient system for the oral, enteral, parenteral, intravenous or topical delivery of mitragynine, mitragynine pseudoindoxyl and/or 7-hydroxymitragynine, and/or other alkaloids, and/or additional drugs to subjects in need thereof. The term “subject” or “patient” refers to a mammal in need of treatment or undergoing treatment using the inventive compounds described herein. Mammalian subjects include without limitation humans, dog, cat, horse or any other animal in need of treatment.

In another embodiment, unilamellar micelles or liposomes that are thermostable at temperatures greater than 50° C. are used in the manufacture of the compound contemplated by this invention. These micelles or liposomes are obtained by contacting a solution of a Mitragyna speciosa plant alkaloids with an appropriate solvent. The mixing of said alkaloid solution occurs in a manner suitable for the rapid dissolution of the alkaloid solution. This can be accomplished through a variety of means including dilution, injection through a small orifice under pressure, and ultrasonic atomization.

And yet in another embodiment, the disclosed compound has advantageous properties, where the micellar and liposomal compound is stable at high temperatures, exceeding 50° C., is stable to sonication, capable of carrying large payloads of Mitragyna speciosa plant alkaloids as well as other drugs suitable for use in combination therapy and can be stored for extended periods of time, for example greater than 20 weeks at 25° C.

In certain embodiments, said compound can be in the form of a concentrated, stable colloidal suspension that is obtained by infusing a solvent solution containing the Mitragyna speciosa plant extract or essentially pure alkaloids into a solvent, with or without buffer. Stabilizing agent, for instance, a polymer or compounds selected from cellulose hyaluronic acid, citric acid, Tris base, sodium carbonate, polyvinyl pyrrolidone (PVP), alginate, chondritin sulfate, poly gamma glutamic acid, gelatin, chitisin, corn starch and flour can be used to stabilize the micelle formulations.

In one embodiment, said compound also exhibits superior systemic delivery and release of Mitragyna speciosa plant alkaloids from the micelle or liposomes used in the manufacture of the contemplated compound. The release of alkaloids from a liposome or micelle of the contemplated compound can be modulated by changing the ratio of the concentration of lipid to the concentration of alkaloids present in the liposome.

In one embodiment, tissue specific delivery can be achieved by modifying the surface of the liposomes or micelles with compounds that bind specifically to biological macromolecules expressed on cellular surfaces. For instance, the micelle or liposomal surface can be derivatized to display an antibody specific to an antigen expressed on cancer cells.

According to one embodiment, said compound that is used in the treatment of a disease condition or other therapy is administered to a patient or subject in need of treatment either alone or in combination with other compounds/drugs having similar or different biological activities. For example, said compound may be administered in a combination therapy, i.e., either simultaneously in single or separate dosage forms or in separate dosage forms within hours or days of each other. Examples of compounds/drugs used in such combination therapies include without limitation: chemotherapeutic agents, immunosuppressive agents, immunostimulatory, anti-pyretic, cytokines, opioids, cannabinoids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

In some embodiments, the compound further contains, in accordance with accepted practices of pharmaceutical compounding, one or more pharmaceutically acceptable excipients, including without limitation: diluents, adjuvants, stabilizers, emulsifiers, preservatives, colorants, buffers, flavor imparting agents. As stated above, said compound may contain Mitragyna speciosa plant alkaloids, their analogs, and co-extraction substances, and may be consumed directly or formulated into nutraceutical or pharmaceutically acceptable compounds suitable for oral, enteral, parenteral, intravenous or topical administration.

The term “parenteral” as used herein includes subcutaneous injections, intravenous, intramuscular, intrasternal injection or infusion techniques. Such excipients are well-known in the art. Dosage forms for oral administration include food, beverages, drinks, soups, baked goods, syrups, oral pharmaceutical compounds, nutraceutical formulations, and the like. Suitable pharmaceutical carriers include any such materials known in the art, e.g., any liquid, gel, solvent, liquid diluent, solubilizer, polymer or the like, which does not significantly interact with other components of the formulations in a deleterious manner.

Liquid dosage forms for oral (peroral) administration include, but are not limited to, pharmaceutically acceptable emulsions, solutions, suspensions, syrups and elixirs. In addition to the Mitragyna speciosa plant extract, the liquid dosage forms can contain inert diluents commonly used in the art. For instance, liquid formulations can contain water, alcohol, polyethylene glycol ethers, and any other pharmaceutically acceptable solvents. Solubilizing agents and emulsifiers such as, without limitation: ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, fatty acid esters of sorbitan and mixtures thereof may also be present in said compound.

Additionally, oral compound of the proposed invention can include, without limitation, adjuvants such as wetting agents, emulsifying and suspending agents, sweetening, flavoring, and perfuming agents. When formulated as a suspension, said compound may contain the Mitragyna speciosa plant extract and suspending agents, for example, without limitation: ethoxylated isostearyl alcohols, polyoxyethylene sorbitol, sorbitan esters, microcrystalline cellulose, aluminum metahydroxide, bentonite, agar-agar, tragacanth, and mixtures thereof.

In one embodiment, the emulsifier may comprise a mixture of monoglyceride and diglyceride at a total concentration of 1% to 99% w/w and a carrageenan or mixture of carrageenans at a total concentration of 0.01% to 10% w/w. In another embodiment, the emulsifier may be present in a concentration range of 1% to 99%, 5% to 80%, 10% to 35%, 10% to 20%, or about 15%-25%% w/w.

Solid dosage forms suitable for oral administration include, capsules, tablets, pills, powders, and granules. In such solid dosage forms, the Mitragyna speciosa plant extract or purified alkaloids can be used alone or in combination with one or more drugs that are mixed with at least one pharmaceutically acceptable excipient or carrier such as sodium citrate or dicalcium phosphate and/or fillers or extenders such as starches, lactose, sucrose, glucose, mannitol, and silicic acid; binders such as, for example, carboxymethylcellulose, alginates, gelatin, polyvinylpyrrolidone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents such as, for example, acetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof. For capsules, tablets and pills, the dosage form can also comprise buffering agents, such as acetic acid and Tris base.

Micellular or liposomal suspensions can be encapsulated with a variety of polymers, sugars, and chelating agents, to yield stable solid preparation. Encapsulation can take the form of cross-linked polymers, trapping of the micells or liposomes within a non-crosslinked polymer network, or dispersed within the crystalline structure of sugar starches or protein molecules. These granules can be further processed to yield sublingual films, suppositories, dispersible powder, tablets, gel capsules, etc.

Solid dosages in the form of tablets, capsules, pills, and granules can be coated using compounds that accelerate or decrease the release of alkaloids. For instance, the proposed invention also encompasses solid dosage forms having enteric coatings, extended-release coatings, sustained-release coatings, delayed release coatings and immediate-release coatings. Methods used to coat solid dosage forms as well as the materials used to manufacture such coatings are well known in the pharmaceutical formulary art. The solid dosage forms can optionally contain opacity enhancing agents. According to one embodiment, the solid dosage form comprises an enteric coating that permits the release of Mitragyna speciosa plant alkaloids or their analogs alone or in combination with one or more drugs, or other Mitragyna speciosa plant alkaloids, at a specific location within the gastrointestinal tract, optionally, in a delayed manner. Exemplary of such coating materials include glyceryl monostearate or glyceryl distearate may be employed, polymeric substances and waxes. The compound contemplated by this invention, alone or in combination with one or more drugs, can also be in micro-encapsulated form, if appropriate, with one or more of the above-mentioned or other excipients.

In one embodiment, said compound is packaged into a gelatin capsule dosage form. In another embodiment, the compound is packaged into a non-gelatin capsule or an HPMC capsule. Said capsule can be a vegan based capsule or else. The compound disclosed herein includes a sustained release compound, an immediate release compound, or a combined sustained release fraction and immediate release fraction. In one embodiment, the therapeutic effect of the compound has a duration up to 4 hours, up to 6 hours, up to 8 hours, up to 10 hours, up to 12 hours, up to 14 hours, up to 16 hours, up to 18 hours, or up to 24 hours. In one embodiment, the compound disclosed herein comprises an immediate release fraction and a sustained release fraction, wherein the immediate release fraction contains a therapeutically effective amount of Mitragyna speciosa plant alkaloids and an edible oil; and wherein the sustained release fraction contains a therapeutically effective amount of Mitragyna speciosa plant alkaloids, and a mixture of emulsifiers and other pharmacologically inactive substances.

In another embodiment, a dietary compound, according to the present invention, is any ingestible preparation that contains the Mitragyna speciosa plant extract as contemplated by this invention, where the pharmacologically inactive substance is a food product. The food product can be dried, cooked, boiled, lyophilized, baked, frozen, chilled, liquid, semi-liquid or prepared by any preparation used in food processing. Such food product can be, but not limited to: breads, teas, soups, cereals, salads, sandwiches, sprouts, vegetables, animal feed, pills and tablets, soft drinks, instant drinks, and any other human or animal food.

In yet another embodiment, a compound for parenteral injection comprises pharmaceutically acceptable sterile aqueous or non-aqueous solutions, dispersions, suspensions or emulsions as well as sterile powders for reconstitution into sterile injectable solutions or dispersions prior to use. Examples of suitable aqueous and non-aqueous carriers, diluents, solvents or vehicles include, without limitation, water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

In one embodiment, proper fluidity can be maintained, for example, by the use of coating materials such as lecithin, by the maintenance of the required particle size in the case of dispersions, and by the use of surfactants. The compound of the present invention can also contain adjuvants such as, but not limited to, preservatives, wetting agents, emulsifying agents, and dispersing agents. The compound for parenteral delivery generally includes isotonic agents such as sugars, sodium chloride, and the like. Prolonged absorption of the injectable pharmaceutical formulation can be brought about by the inclusion of agents which delay absorption such as aluminum monostearate and gelatin.

Injectable depot forms are made, in one embodiment, by forming microencapsule matrices of the drug in biodegradable polymers such as polylactide-polyglycolide. Depending upon the ratio of drug to polymer and the nature of the specific polymer employed, the rate of drug release can be controlled. Examples of other biodegradable polymers include poly-orthoesters and poly-anhydrides. Depot injectable formulations are also prepared by entrapping the drug in liposomes or microemulsions which are compatible with body tissues. The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Dosage forms for topical administration include, but are not limited to, ointments, creams, emulsions, lotions, gels, sunscreens and agents that favor penetration within the epidermis. Various additives, known to those skilled in the art, may be included in the topical formulations of the invention. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil.

Suitable skin permeation enhancers are well known in the art and include lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C2-C6 alkanediols; dimethyl formamide (DMF), N,N-dimethylacetamide (DMA) and tetrahydrofurfuryl alcohol. Examples of solubilizers include, but are not limited to, hydrophilic ethers such as diethylene glycol monoethyl ether (ethoxydiglycol, available commercially as Transcutol) and diethylene glycol monoethyl ether oleate (available commercially as Softcutol); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol); alkyl methyl sulfoxides, such as DMSO; pyrrolidones, DMA, and mixtures thereof.

Prevention and/or treatment of infections can be achieved by the inclusion of antibiotics, as well as various antibacterial and antifungal agents, for example, paraben, chlorobutanol, phenol sorbic acid, and the like, in the compounds of the invention.

One of ordinary skill will appreciate that effective amounts of the agents in the compound used in the methods of the invention can be determined empirically. It will be understood that, when administered to a patient, the total daily usage of the compound of the present invention will be decided by the attending physician within the scope of sound medical judgment. The specific therapeutically effective dose level for any patient will depend upon a variety of factors: the type and degree of the response to be achieved; the activity of the specific compound employed; the age, body weight, general health, sex and diet of the patient; the duration of the treatment; drugs used in combination or coincidental with the method of the invention; and like factors well known in the medical arts.

Mitragynine (FIG. 4 (1)) is the most abundant alkaloid in the leaves. It was first isolated in 1921 and its chemical structure was fully elucidated in 1964. The systematic (Chemical Abstract) name is (αE,2S,3S,12bS)-3-ethyl-1,2,3,4,6,7,12,12b-octahydro-8-methoxy-α-(methoxymethylene)-indolo[2,3-a]quinolizine-2-acetic acid methyl ester (CAS Registry Number: 4098-40-2). Other names: (E)-16,17-didehydro-9,17-dimethoxy-17,18-seco-20α-yohimban-16-carboxylic acid methyl ester, 9-methoxycorynantheidine, and SK&F 12711. Mitragynine is poorly soluble in water but soluble in conventional organic solvents, including acetone, acetic acid, alcohols, chloroform and diethyl ether providing fluorescent solutions. Mitragynine distils at 230-240° C. at 5 mmHg. It forms white, amorphous crystals that melt at 102-106° C. The melting point of mitragynine hydrochloric acid salt is 243° C.; the picrate melts at 223-224° C. and the acetate at 142° C.

The 7-Hydroxymitragynine (FIG. 4 (2)) is present in small amounts in Mitragyna speciosa leaves and was identified in 1993. Its systematic (Chemical Abstract) name is (αE,2S,3S,7aS,12bS)-3-ethyl-1,2,3,4,6,7,7a,12b-octahydro-7a-hydroxy-8-methoxy-α-(methoxymethylene)-indolo[2,3-a]quinolizine-2-acetic acid methyl ester (CAS Registry Number: 174418-82-7). On average, Mitragyna speciosa plant extract contains less than 2% of said substance. Mitragynine pseudoindoxyl (FIG. 4 (3)) is a rearrangement product of 7-hydroxymitragynine. Its systemic name is Methyl (E)-2-((1'S,6'S,7'S,8a'S)-6′-ethyl-4-methoxy-3-oxo-3′,5′,6′,7′,8′,8a′-hexahydro-2′H-spiro[indoline-2,1′-indolizin]-7′-yl)-3-methoxyacrylate.

The formulations of the invention, in one embodiment, are particularly suitable for oral administration and may be administered to subjects with a new or pre-existing condition or pre-disposed to certain disease conditions, or under certain circumstances, such as without limitation: injury, illness or abnormal function of peripheral or central nervous system; chronic nerve pain; spinal neuropathic pain; supraspinal neuropathic pain; central neuropathic pain; peripheral neuropathic pain, sympathetic pain; postherpetic neuralgia; root avulsions; painful traumatic mononeuropathy; painful polyneuropathy; central pain syndromes; postsurgical pain syndromes; complex regional pain syndrome; tooth or facial nerve pain; typical and atypical odontalgia; allodynia; abnormally activated neuronal circuits, chemical imbalance, mental health disorder, emotional stress, major depression; dysthymia; postpartum depression; seasonal affective disorder; atypical depression; psychotic depression; bipolar disorder; premenstrual dysphoric disorder; situational depression; persistent depressive disorder.

In one embodiment of the proposed invention, the following types of peripherally initiated neuropathic pain can be treated mitragynine, mitragynine pseudoindoxyl and 7-hydroxymitragynine: carpal tunnel syndrome, meralgia paresthetica, diabetic neuropathy, alcohol/nutritional neuropathy, acute/subacute radicular pain (L5/S1 C6/C7), trigeminal neuralgia, trigeminal neuropathy (atypical facial pain, anesthesia dolorosa, post-traumatic neuropathy), postherpetic neuralgia, failed back syndrome (root sleeve fibrosis, arachnoiditis), brachial plexus neuropathies/avulsion, incisional neuralgia (postmastectomy pain syndrome, post-thoracotomy pain), causalgia guillain bane syndrome, phantom limb pain.

In another embodiment of the proposed invention, the following types of central pain syndromes can be treated mitragynine, mitragynine pseudoindoxyl and 7-hydroxymitragynine: thalamic syndrome (stroke, tumour), multiple sclerosis (trigeminal neuralgia, painful tonic seizures), wallenberg's (lateral medullary) syndrome, syringomyelia post-traumatic central pain.

In one embodiment, post-lumbar surgery patient that developed chronic back pain underwent a microdiscectomy at L4/L5. Shortly after the surgery, the patient developed severe pain and was diagnosed with adhesive arachnoiditis (AA) by MRI. The cause of the AA was likely the placement of a corticosteroid directly into the surgical site. To relieve the patient's pain, he was started on a regimen of opioid drugs. The patient's pain management regimen included: (1) fentanyl transdermal patch, 100 mcg every other day; (2) transmucosal fentanyl, 800 mcg 8 times per day; and (3) duloxetine (Cymbalta) 60 mg per day. After development of analgesic resistance, the treatment became marginally effective. The patient described his pain as being from the waist down and that it was an intense, burning sensation. The proposed initial clinical treatment that included the novel substances proposed herein, in one embodiment would be amending his analgesic regimen as follows: (1) acetazolamide (Diamox) 500 mg daily, which is approved to treat glaucoma, to lower pressure within the spinal cord; (2) peroral 7-hydroxymitragynine 8 mg every 4-6 hours for breakthrough pain; (4) peroral mitragynine 100 mg every 8 hours; (5) duloxetine (Cymbalta) 60 mg per day; and (3) topical morphine massaged over the site of his adhesions.

In another embodiment, a 28-year-old man showed clumping of the nerve rootlets of the cauda equina at the L-3 level. The diagnostic imaging also showed degenerative changes in the cervical and lumbar spines. The patient was diagnosed with post-traumatic neuropathy and was begun on opioid medications for pain relief. He has been on opioids for more than 15 years. His medication regimen included oxycodone extended release, 80 mg 9 times per day—total daily dose: 720 mg; hydromorphone immediate release, 80 mg per day; and fentanyl transmucosal, 1600 mcg twice daily. Despite this regimen, the patient was bed-bound most days and experienced increased pain with walking, lifting, bending over, and lying down for more than 2 hours. The patient was taught gentle stretching exercises of the extremities and spine, and was started on mitragynine pseudoindoxyl extended release, 20 mg 4 times per day, and (2) 7-hydroxymitragynine immediate release, 10 mg, taken 4-6 times a day for breakthrough pain.

In one embodiment of the proposed invention, the treatment of spasticity and pain related to an autoimmune disease involves giving to a patient every 12 hours on an empty stomach by oral administration a one soft-gel capsule of the compound containing 10 mg of 7-hydroxymitragynine (FIG. 5); and in another embodiment, 100 mg of mitragynine; and in another embodiment, 20 mg of mitragynine pseudoindoxyl; and in another embodiment a number of pharmacologically inactive substances, such as a lipid carrier. Said capsule is an extended release and time-release capsule designed to release said mixture in the small intestine; and in another embodiment, in the stomach. The aforesaid compound and method in some subjects may reduce neurological pain and spasticity symptoms associated with, in one embodiment, with multiple sclerosis. In another embodiment, said compound is administered in conjunction with a therapeutic dose of gabapentin or another substance, where the medication is taken at bed time.

In one embodiment, a mixture of methadone isomers was used in combination with (−)-mitragynine, demonstrating greater efficacy in the treatment of pain than an equivalent dose on a molar basis than either of the individual active compounds by themselves. For example, a mixture comprising of five moles of methadone isomers and one mole of (−)-mitragynine and/or (+)-mitragynine, and/or (z)-mitragynine. The mixture has greater efficacy and significantly lower side effects. For example, in mice, constipation was reduced by more than 30% compare to the methadone alone. Development of tolerance after 30 days of administration was also reduced by at least 25% compare to the methadone alone.

In another embodiments, 7-hydroxymitragynine is used in the treatment of neuropathic in combination with a calcium channel binding agent, such as gabapentin, pregabalin, or gabapentin enacarbil, which exhibit a synergistic efficacy in the treatment of neuropathic pain compared to equivalent doses of the opioid compound alone or the calcium channel binding agent alone.

In another embodiment, 7-hydroxymitragynine exhibits efficacy in the treatment of mixed pain states, i.e. a combination of neuropathic pain and nociceptive pain. In still another embodiment, a 7-hydroxymitragynine analog MGM 15 or MGM 16 exhibits efficacy in the treatment of mixed pain states. And in another embodiment, a 7-hydroxymitragynine analog that is also at least a partial agonist of DOR exhibits efficacy in the treatment of mixed pain states.

The potential commercial uses of the disclosed preparations include, for example, protective/prophylactic and medical uses. The compounds of the invention can also be administered by a variety of other routes, including mucosal, subcutaneous and intramuscular administration, and may comprise a variety of carriers or excipients known in the formulary art, such as, non-toxic solid, semisolid or liquid filler, diluent, encapsulating material and formulation auxiliaries that are pharmaceutically acceptable.

The features disclosed in the foregoing description, or the following claims, or the accompanying drawings, expressed in their specific forms or in terms of a means for performing the disclosed function, or a method or system for attaining the disclosed result, as appropriate, may separately, or in any combination of such features, be utilized for realizing the invention in diverse forms thereof.

While various embodiments of the present invention have been described above, it should be understood that they have been presented by way of example only, and not limitation. It will be understood by those skilled in the art that various changes in form and details may be made therein without departing from the spirit and scope of the invention as defined in the appended claims. Thus, the breadth and scope of the present invention should not be limited by any of the above-described exemplary embodiments but should be defined in accordance with the following claims and their equivalent.

### REFERENCES

Abdelhamid, E. E., Sultana, M., Portoghese, P. S., & Takemori, A. E. (1991, Jul. 1). Selective blockage of delta opioid receptors prevents the development of morphine tolerance and dependence in mice. J Pharmacol Exp Ther., 258(1), pp. 299-303. Retrieved from https://www.ncbi.nlm.nih.gov/pubmed/1649297
Abushwereb, H. S., Abdulatif, A., & Abdulmajeed, A. (2018, May). The Antidepressant-like Effect of Mitragyna Speciosa Korth. J. Pharm. Appl., 4(2), pp. 109-113. doi:10.18576/jpac/040205
Amorim, D. (2015). Pharmacological treatment of neuropathic pain: review of oral and topical therapy recommendations. International Journal of Clinical Neurosciences and Mental Health (2), pp. 1-9. doi:10.21035/ijcnmh.2015.2.4
Arnér, S., & Meyerson, B. A. (1988, April). Lack of analgesic effect of opioids on neuropathic and idiopathic forms of pain. Pain, 33(1), pp. 11-23. Retrieved from https://www.ncbi.nlm.nih.gov/pubmed/2454440
Bohn, L. M., Gainetdinov, R. R., Lin, F., & et al. (2000, Dec. 7). μ-Opioid receptor desensitization by β-arrestin-2 determines morphine tolerance but not dependence. Nature, 408(6813), pp. 720-723. doi:10.1038/35047086
Bohn, L. M., Lefkowitz, R. J., Gainetdinov, R. R., & et al. (1999, Dec. 24). Enhanced Morphine Analgesia in Mice Lacking β-Arrestin 2. Science, 286(5449), pp. 2495-2498. doi:10.1126/science.286.5449.2495
Bu, H., Liu, X., Tian, X., & et al. (2015, January). Enhancement of morphine analgesia and prevention of morphine tolerance by downregulation of β-arrestin 2 with antigene RNAs in mice. Int J Neurosci., 125(1), pp. 56-65. doi:10.3109/00207454.2014.896913
Carr, G. V., Bangasser, D., Bethea, T., & et al. (2009, November). Antidepressant-Like Effects of Kappa-Opioid Receptor Antagonists in Wistar Kyoto Rats. Neuropsychopharmacology 35(3):752-63, 35(3), pp. 752-63. doi:10.1038/npp.2009.183
Chittrakarn, S., Keawpradub, N., Sawangjaroen, K., Kansenalak, S., & Benjamas, J. (2010). The neuromuscular blockade produced by pure alkaloid, mitragynine and methanol extract of kratom leaves (Mitragyna speciosa Korth.). Journal of Ethnopharmacology, 129, 344-349. Retrieved from http://entheology.com/wp-content/uploads/kratom-research/2010_std008.pdf
Dahan, A., Sarton, E., Teppema, L., & et al. (2001, May). Anesthetic Potency and Influence of Morphine and Sevoflurane on Respiration in μ-Opioid Receptor Knockout Mice. (5, Ed.) Anesthesiology, 94, pp. 824-832. Retrieved from https://www.ncbi.nlm.nih.gov/pubmed/11388534
DeWire, S. M., Yamashita, D. S., Rominger, D. H., & et al. (2013, March). A G protein-biased ligand at the μ-opioid receptor is potently analgesic with reduced gastrointestinal and respiratory dysfunction compared with morphine. J Pharmacol Exp Ther., 344(3), pp. 708-17. doi:10.1124/jpet.112.201616
Finnerup, N. B., Sindrup, S. H., & Jensen, T. S. (2010, September). The Evidence for Pharmacological Treatment of Neuropathic Pain. Pain, 150(3), pp. 573-81. doi:10.1016/j.pain.2010.06.019
Gagnon, B., Almahrezi, A., & Schreier, G. (2003, Fall). Methadone in the treatment of neuropathic pain. Pain Res Manag., 8(3), pp. 149-54. Retrieved from https://www.ncbi.nlm.nih.gov/pubmed/14657982
Gassaway, M. M., Rives, M. L., Kruegel, A. C., Javitch, J. A., & Sames, D. (2014, July). The atypical antidepressant and neurorestorative agent tianeptine is a μ-opioid receptor agonist. Transl Psychiatry, 4(7), pp. 1-5. doi:10.1038/tp.2014.30
Gaveriaux-Ruff, C., Nozaki, C., & Nadal, X. (2011, June). Genetic ablation of delta opioid receptors in nociceptive sensory neurons increases chronic pain and abolishes opioid analgesia. Pain, 152(6), pp. 1238-48. doi:10.1016/j.pain.2010.12.031
Groer, C. E., Tidgewell, K., Moyer, R. A., & et al. (2007, February). An opioid agonist that does not induce mu-opioid receptor—arrestin interactions or receptor internalization. Mol Pharmacol., 71(2), pp. 549-57. doi:10.1124/mol.106.028258
Hepburn, M. J., Little, P. J., Gingras, J., & Kuhn, C. M. (1997, June). Differential effects of naltrindole on morphine-induced tolerance and physical dependence in rats. J Pharmacol Exp Ther., 281(3), pp. 1350-6. Retrieved from https://www.ncbi.nlm.nih.gov/pubmed/9190871
Holaday, J. W., Hitzemann, R. J., Curell, J., & et al. (1982, November). Repeated electroconvulsive shock or chronic morphine treatment increases the number of 3H-D-ALA2, D-LEU3-enkephalin binding sites in rat brain membranes. Life Sciences, 3/(20-21), pp. Life Sciences, Vol. 31, pp. 2359-2362. doi:https://doi.org/10.1016/0024-3205(82)90156-4
Houghton, P. J., Latiff, A., & Said, I. M. (1991). Alkaloids from Mitragyna speciosa. Phytochemistry, 30, 347-350.
Idayu, N. F., Hidayat, M. T., Moklas, M. A., & et al. (2011, Mar. 15). Antidepressant-like effect of mitragynine isolated from Mitragyna speciosa Korth in mice model of depression. Phytomedicine, 18(5), pp. 402-7. doi:10.1016/j.phymed.2010.08.011
Kest, B., Lee, C. E., McLemore, G. L., & Inturrisi, C. E. (1996). An antisense oligodeoxynucleotide to the delta opioid receptor (DOR-1) inhibits morphine tolerance and acute dependence in mice. Brain Res Bull., 39(3), pp. 185-8. Retrieved from https://www.ncbi.nlm.nih.gov/pubmed/8866695
Kruegel, A. C., Gassaway, M. M., & Kapoor, A. (2016, Jun. 1). Synthetic and Receptor Signaling Explorations of the Mitragyna Alkaloids: Mitragynine as an Atypical Molecular Framework for Opioid Receptor Modulators. J Am Chem Soc., 138(21), pp. 6754-64. doi:10.1021/jacs.6b00360
Kumarnsit, E., Keawpradub, N., & Nuankaew, W. (2007, April). Effect of Mitragyna speciosa aqueous extract on ethanol withdrawal symptoms in mice. Fitoterapia., 78(3), pp. 182-5. doi:10.1016/j.fitote.2006.11.012
Kumarnsit, E., Vongvatcharanon, U., Keawpradub, N., & Intasaro, P. (2007, Apr. 12). Fos-like immunoreactivity in rat dorsal raphe nuclei induced by alkaloid extract of Mitragyna speciosa. Neurosci Lett., 416(2), pp. 128-32. doi:10.1016/j.neulet.2007.01.061
Lamb, K., Tidgewell, K., Simpson, D. S., & et al. (2012, Mar. 1). Antinociceptive Effects of Herkinorin, a MOP Receptor Agonist Derived from Salvinorin A in the Formalin Test in Rats: New Concepts in Mu Opioid Receptor Pharmacology. Drug Alcohol Depend., 121(3), pp. 181-188. doi:10.1016/j.drugalcdep.2011.10.026
Li, Y., Liu, X., Liu, C., & et al. (2009). Improvement of Morphine-Mediated Analgesia by Inhibition of β-Arrestin 2 Expression in Mice Periaqueductal Gray Matter. Int. J. Mol. Sci., 10(3), pp. 954-963. doi:10.3390/ijms10030954
Li, W., Sun, H., Chen, H., Yang, X., & et al. (2016). Major Depressive Disorder and Kappa Opioid Receptor Antagonists. Transl Perioper Pain Med., 1(2), pp. 4-16. Retrieved from https://www.ncbi.nlm.nih.gov/pmc/articles/PMC4871611
Ma, J., Yin, W., Zhou, H., & Cook, J. M. (2007). Total Synthesis of the Opioid Agonistic Indole Alkaloid, Mitragynine, as well as the First Total Synthesis of 9-Methoxygeissoschizol and 9-Methoxy-Nb-methylgeissoschizol. Bethesda: U.S. National Library of Medicine. doi:10.1021/o10712201
Manda, V., Avula, B., & Ali, Z. (2014, May). Evaluation of In Vitro Absorption, Distribution, Metabolism, and Excretion (ADME) Properties of Mitragynine, 7-Hydroxymitragynine, and Mitraphylline. Planta Medica, 80(7), pp. 568-76. doi:10.1055/s-0034-1368444
Mansikka, H., Zhao, C., Sheth, R. N., & et al. (2004, April). Nerve Injury Induces a Tonic Bilateral μ-Opioid Receptor-mediated Inhibitory Effect on Mechanical Allodynia in Mice. Anesthesiology 4 2004, Vol. 100, 912-921, 100(4), pp. 912-921. doi:10.1097/00000542-200404000-00022
Matsumoto, K., Narita, M., Muramatsu, N., & et al. (2014, March). Orally Active Opioid μ/δ Dual Agonist MGM-16, a Derivative of the Indole Alkaloid Mitragynine, Exhibits Potent Antiallodynic Effect on Neuropathic Pain in Mice. Journal of Pharmacology and Experimental Therapeutics, 348(3), pp. 383-392. doi:10.1124/jpet.113.208108
Matthes, H., Maldonado, R., Simonin, F., & et al. (1996, Oct. 31). Loss of morphine-induced analgesia, reward effect and withdrawal symptoms in mice lacking the μ-opioid-receptor gene. Nature, 383(6603), pp. 819-23. doi:10.1038/383819a0
Miyamoto, Y., Portoghese, P. S., & Takemori, A. E. (1993, March). Involvement of delta 2 opioid receptors in the development of morphine dependence in mice. Journal of Pharmacology and Experimental Therapeutics, 264(3), pp. 1141-1145. Retrieved from http://jpet.aspetjournals.org/content/264/3/1141
Nadal, X., Baños, J. E., Kieffer, B. I., & Maldonado, R. (2006, Feb. 23). Neuropathic pain is enhanced in delta-opioid receptor knockout mice. Eur J Neurosci., 23(3), pp. 830-4. doi:10.1111/j.1460-9568.2006.04569.x
Obata, H. (2017, Nov. 21). Analgesic Mechanisms of Antidepressants for Neuropathic Pain. Int J Mol Sci., p. pii: E2483. doi:10.3390/ijms18112483
Ossipov, M. H., Kovelowski, C. J., & Porreca, F. (1995, Jan. 30). The increase in morphine antinociceptive potency produced by carrageenan-induced hindpaw inflammation is blocked by naltrindole, a selective δ-opioid antagonist. Neuroscience Letters, 184(3), pp. 173-176. doi:10.1016/0304-3940(94)11199-S
Raehal, K. M., Schmid, C. L., Groer, C. E., & Bohn, L. M. (2011, December). Functional Selectivity at the μ-Opioid Receptor: Implications for Understanding Opioid Analgesia and Tolerance. Pharmacological Reviews, 63(4), pp. 1001-1019. doi:10.1124/pr.111.004598
Raja, S. N., Haythornthwaite, J. A., Pappagallo, M., & et al. (2002, Oct. 8). Opioids versus antidepressants in postherpetic neuralgia: a randomized, placebo-controlled trial. Neurology, 59(7), pp. 1015-21. doi:10.1212/WNL.59.7.1015
Rothman, R. B., Danks, J. A., Jacobson, A. E., & et al. (1986, May 13). Morphine tolerance increases μ-noncompetative δ binding sites. European Journal of Pharmacology, 124(1-2), pp. 113-119. doi:10.1016/0014-2999(86)90130-5
Rowbotham, M. C., Twilling, L., Davies, P. S., & et al. (2003, April). Oral Opioid Therapy for Chronic Peripheral and Central Neuropathic Pain. New England Journal of Medicine, 348(13), pp. 1223-32. doi:10.1056/NEJMoa021420
Salvat, E., Yalcin, I., Muller, A., & Barrot, M. (2018). A comparison of early and late treatments on allodynia and its chronification in experimental neuropathic pain. Molecular Pain, 14, pp. 1-10. doi:10.1177/1744806917749683
Schmid, C. L., Kennedy, N. M., Ross, N. C., & et al. (2017, Nov. 16). Bias Factor and Therapeutic Window Correlate to Predict Safer Opioid Analgesics. Cell, 171(5), pp. 1165-1175.e13. doi:10.1016/j.ce11.2017.10.035
Shellard, E. J., Houghton, P. J., & Resha, M. (1978). The Mitragyna species of Asia. Part XXXI. The alkaloids of Mitragyna speciosa Korth from Thailand. Planta Medica, 34, 26-36.
Smith, H. S. (2012, July). Opioids and Neuropathic Pain. Pain Physician., 15(3 Suppl.), pp. ES93-110. Retrieved from https://www.ncbi.nlm.nih.gov/pubmed/22786465
Soergel, D. G., Subach, R. A., Burnham, N., & et al. (2014, September). Biased agonism of the μ-opioid receptor by TRV130 increases analgesia and reduces on-target adverse effects versus morphine: A randomized, double-blind, placebo-controlled, crossover study in healthy volunteers. Pain, 155(9), pp. 1829-35. doi:10.1016/j.pain.2014.06.011
Stein, C. (1995, Jun. 22). The control of pain in peripheral tissue by opioids. N Engl J Med., 332(25), pp. 1685-90. doi:10.1056/NEJM199506223322506
Suhanya, P., Ramanathan, S., Ismail, S., & et. al. (2010, July). Determination of mitragynine in plasma with solid-phase extraction and rapid HPLC-UV analysis, and its application to a pharmacokinetic study in rat. Analytical and Bioanalytical Chemistry, 397, pp. 2023-2030. doi:10.1007/s00216-010-3707-7
Suzuki, T., Tsuji, M., Mori, T., Misawa, M., & Nagase, H. (1997, May-June). Involvement of delta 1 and delta 2 opioid receptor subtypes in the development of physical dependence on morphine in mice. Pharmacol Biochem Behav., 57(1-2), pp. 293-9. Retrieved from https://www.ncbi.nlm.nih.gov/pubmed/9164585
Traynor, J. R., & Elliott, J. (1993, Mar. 14). delta-Opioid receptor subtypes and cross-talk with mu-receptors. Trends Pharmacol Sci., 14(3), pp. 84-6. Retrieved from https://www.ncbi.nlm.nih.gov/pubmed/8387705
Trevena, Inc. (2018). Trevena, Inc. Retrieved from Oliceridine injection: http://www.trevena.com/Oliceridine.php
Trevena, Inc. (2018). Trevena, Inc. Retrieved from TRV734: http://www.trevena.com/TRV734.php
Watson, C. P., & Babul, N. (1998, June). Efficacy of oxycodone in neuropathic pain: a randomized trial in postherpetic neuralgia. Neurology, 50(6), pp. 1837-41. doi:10.1212/WNL.50.6.1837
Watson, C. P., & Watt-Watson, J. H. (1999, March 19). Treatment of Neuropathic Pain: Focus on Antidepressants, Opioids and Gabapentin. Pain Research and Management, 4(4), pp. 168-178. doi:10.1155/1999/961273
Zhao, M., Wang, J. Y., Jia, H., & Tang, J. S. (2006, Mar. 3). mu-but not delta- and kappa-opioid receptors in the ventrolateral orbital cortex mediate opioid-induced antiallodynia in a rat neuropathic pain model. Brain Res., 1076(1), pp. 68-77. doi:10.1016/j.brainres.2006.01.018
Zhu, Y., King, M. A., Schuller, A. G., & et al. (1999, September). Retention of supraspinal delta-like analgesia and loss of morphine tolerance in delta opioid receptor knockout mice. Neuron., 24(1), pp. 243-52. Retrieved from https://www.ncbi.nlm.nih.gov/pubmed/10677041
Zorn, K. E., & Fudin, J. (2011, May). Treatment of Neuropathic Pain: The Role of Unique Opioid Agents. Practical Pain Management, 11(4), pp. 26-33. Retrieved from https://www.practicalpainmanagement.com/treatment-neuropathic-pain-role-unique-opioid-agents
[symbol] (2009). CN Patent No. 102048857B. Retrieved from https://www.google.com/patents/CN102048857A

What claimed is:

1. A method for treating neuropathic pain in subjects in need thereof; the method comprises of administering to the subject 1 mg or more of a compound, one or more times in 24 hours, that contains not less than 6% of mitragynine by mass, or not less than 3% of mitragynine pseudoindoxyl by mass, or not less than 3% of 7-hydroxymitragynine by mass; and where said compound contains a trace amount or more of one or more other substances.

2. The method of claim 1, where said compound contains a trace amount or more of one or more of the following: morphine; methadone; su-, remi- or fentanyl; tramadol; oxycodone; hydrocodone; buprenorphine; meperidine; typical or atypical opioid compound; selective serotonin reuptake inhibitor compound; monoamine oxidase inhibitor compound; tricyclic antidepressant compound; serotonin and norepinephrine reuptake inhibitor compound; atypical antidepressant compound; nonsteroidal anti-inflammatory compound; local anesthetic compound; non-narcotic analgesic compound; one or more other of plurality of indole or oxindole alkaloids; one or more cannabinoids; or a combination thereof.

3. The method of claim 1, where said compound is administered in one of the following forms: sustained release, extended release, combined sustained release and extended release dosage forms, immediate release dosage forms, combined sustained release and immediate release dosage forms, gastric release dosage forms, intestinal release dosage forms, or a combination thereof; and where said compound is administered in a dosage form selected from the group consisting of: a tablet, a liquid dosage form, a hard gelatin capsule, a soft gelatin capsule, an HPMC capsule, an inhalant, an injectable, a transdermal, a buccal, a sublingual, and a rectal or a vaginal suppository; and where said compound is administered by a route selected from the group consisting of: peroral, intranasal, inhalation, parenteral, transdermal, rectal, vaginal, buccal, sublingual, or combination thereof.

4. The method of claim 1, where said compound is administered in combination with one or more other medications, dietary supplements, and/or foods.

5. The method of claim 1, where said treatment or therapy involves treatment or therapy of one or more of the following: injury, illness or abnormal function of peripheral or central nervous system; chronic nerve pain; spinal neuropathic pain; supraspinal neuropathic pain; central neuropathic pain; peripheral neuropathic pain, sympathetic pain; postherpetic neuralgia; root avulsions; painful traumatic mononeuropathy; painful polyneuropathy; central pain syndromes; postsurgical pain syndromes; complex regional pain syndrome; tooth or facial nerve pain; typical and atypical odontalgia; allodynia; or a combination thereof.

6. The method of claim 1, where said treatment or therapy involves treatment or therapy of one or more of the following causes of nerve pain: toxicity; metabolism abnormality; trauma; compression; autoimmune abnormality; congenital/hereditary abnormality; infection, or a combination thereof.

7. The method of claim 1, where said mitragynine, or said 7-hydroxymitragynine, or said mitragynine pseudoindoxyl are one or more of: natural substances that have been purified or modified; synthetically derived substances; semi-synthetic substances; esterified substances or salts; active metabolites of any of the foregoing, pro-drugs of any of the foregoing; analogs and isomers of any of the foregoing; derivatives of any of the foregoing; suspensions of any of the forgoing; and mixtures thereof.

8. A compound for treating neuropathic pain in subjects in need thereof; the compound that contains not less than 6% of mitragynine by mass, or not less than 3% of mitragynine pseudoindoxyl by mass, or not less than 3% of 7-hydroxymitragynine by mass; and where said compound contains a trace amount or more of one or more other substances.

9. The compound of claim 8, where said compound contains a trace amount or more of one or more of: morphine; methadone; su-, remi- or fentanyl; tramadol; oxycodone; hydrocodone; buprenorphine; meperidine; typical or atypical opioid compound; selective serotonin reuptake inhibitor compound; monoamine oxidase inhibitor compound; tricyclic antidepressant compound; serotonin and norepinephrine reuptake inhibitor compound; atypical antidepressant compound; nonsteroidal anti-inflammatory compound; local anesthetic compound; non-narcotic analgesic compound; one or more other of plurality of indole or oxindole alkaloids; one or more cannabinoids; or a combination thereof.

10. The compound of claim 8, where said treatment or therapy involves treatment or therapy of at least one of: injury, illness or abnormal function of peripheral or central nervous system; chronic nerve pain; spinal neuropathic pain; supraspinal neuropathic pain; central neuropathic pain; peripheral neuropathic pain, sympathetic pain; postherpetic neuralgia; root avulsions; painful traumatic mononeuropathy; painful polyneuropathy; central pain syndromes; postsurgical pain syndromes; complex regional pain syndrome; tooth or facial nerve pain; typical and atypical odontalgia; allodynia; or a combination thereof.

11. The compound of claim 8, where said treatment or therapy involves treatment or therapy of one or more of the following causes of nerve pain: toxicity; metabolism abnormality; trauma; compression; autoimmune abnormality; congenital/hereditary abnormality; infection, or a combination thereof.

12. The compound of claim 8, where said mitragynine, or said 7-hydroxymitragynine, or said mitragynine pseudoindoxyl are one or more of: natural substances that have been purified or modified; synthetically derived substances; semi-synthetic substances; esterified substances or salts; active metabolites of any of the foregoing, pro-drugs of any of the foregoing; analogs and isomers of any of the foregoing; derivatives of any of the foregoing; suspensions of any of the forgoing; and mixtures thereof.

13. The compound of claim 8, where said mitragynine or said 7-hydroxymitragynine is derived from Mitragyna speciosa plant.

14. The compound of claim 8, where said compound is packaged in a dosage form selected from the group consisting of: sustained release, extended release, combined sustained release and extended release dosage forms, immediate release dosage forms, combined sustained release and immediate release dosage forms, gastric release dosage forms, intestinal release dosage forms, or a combination thereof; and where said compound is administered in a dosage form selected from the group consisting of: a tablet, a liquid dosage form, a hard gelatin capsule, a soft gelatin capsule, an HPMC capsule, an inhalant, an injectable, a transdermal, a buccal, a sublingual, and a rectal or a vaginal suppository; and where said compound is administered by a route selected from the group consisting of: peroral, intranasal, inhalation, parenteral, transdermal, rectal, vaginal, buccal, sublingual, or combination thereof.

15. The compound of claim 8, where said one or more other substances is one or more of the following: pharmaceutically acceptable oil; lipid carrier; polymer; stabilizing agent; diluent; adjuvant; emulsifier; preservative; colorant; flavor imparting agent; acid; Tris base; sodium carbonate, or a combination thereof.

16. The compound of claim 8, where said compound contains one or more other medications or dietary supplements, or food products, or flavonoids, or terpenoid saponins, or polyphenols, or glycosides, or a combination thereof.

17. The compound of claim 8, where said mitragynine, or 7-hydroxymitragynine, or mitragynine pseudoindoxyl is dispersed in an inert water-soluble carrier at a solid state (solid dispersion), and/or incorporated into a lipid carrier.

18. The compound of claim 8, where the initial purity of said mitragynine or said 7-hydroxymitragynine, or said mitragynine pseudoindoxyl by HPLC is 70% or more by area and the stability is such that 50% or more by area remains in undegraded form after exposure of the compound to the storage conditions for twelve months or more, where the ambient temperature is between 20° C. and 40° C. and relative humidity is between 55% and 75%.

19. A method for treating depression in subjects in need thereof; the method comprises of administering to the subject 1 mg or more of a compound, one or more times in 24 hours, that contains not less than 1% of mitragynine pseudoindoxyl by mass, or not less than 1% of 7-hydroxymitragynine by mass; and where said compound contains a trace amount or more of one or more other substances.

20. A compound for treating depression in subjects in need thereof; the compound contains not less than 1% of mitragynine pseudoindoxyl by mass, or not less than 1% of 7-hydroxymitragynine by mass; and where said compound contains a trace amount or more of one or more other substances.

21. A method for treating acute and chronic pain in subjects in need thereof; the method comprises of administering to the subject 1 mg or more of a compound, one or more times in 24 hours, that contains not less than 1% of mitragynine by mass, or not less than 0.1% of mitragynine pseudoindoxyl by mass, or not less than 0.1% of 7-hydroxymitragynine by mass; and where said compound contains a trace amount or more of one or more substances that provide at least partial activation of mu-opioid receptors or CB1 or CB2 cannabinoid receptors.

22. A compound for treating acute and chronic pain in subjects in need thereof; the compound contains not less than 1% of mitragynine by mass, or not less than 1% of mitragynine pseudoindoxyl by mass, or not less than 1% of 7-hydroxymitragynine by mass; and where said compound contains a trace amount or more of one or more substances that provide at least partial activation of mu-opioid receptors or CB1 or CB2 cannabinoid receptors.

23. A compound for treating neuropathic pain and depression in subjects in need thereof, where the compound contains not less than 1% by mass of one or more substances that exhibit in human subjects:
a) at least partial activation of mu-opioid receptors without recruiting β-arrestin; and
b) at least partial blocking of delta-opioid receptors; and
c) at least partial activation of α2-adrenoceptors; and/or
d) at least partial blocking or downregulating of 5-HT1A and/or 5-HT2A receptors; and
where said compound contains a trace amount or more of one or more other substances.

24. The method and compound of claims 19, 20, 21, 22 and 23, where said treatment or therapy involves treatment or therapy of at least one of: injury, illness or abnormal function of peripheral or central nervous system; chronic nerve pain; spinal neuropathic pain; supraspinal neuropathic pain; central neuropathic pain; peripheral neuropathic pain, sympathetic pain; postherpetic neuralgia; root avulsions; painful traumatic mononeuropathy; painful polyneuropathy; central pain syndromes; postsurgical pain syndromes; complex regional pain syndrome; tooth or facial nerve pain; typical and atypical odontalgia; allodynia; abnormally activated neuronal circuits, chemical imbalance, mental health disorder, emotional stress, major depression; dysthymia; postpartum depression; seasonal affective disorder; atypical depression; psychotic depression; bipolar disorder; premenstrual dysphoric disorder; situational depression; persistent depressive disorder; or a combination thereof.

25. The compound of claims 20, 22 and 23, where said compound is packaged in a dosage form selected from the group consisting of: sustained release, extended release, combined sustained release and extended release dosage forms, immediate release dosage forms, combined sustained release and immediate release dosage forms, gastric release dosage forms, intestinal release dosage forms, or a combination thereof and where said compound is administered in a dosage form selected from the group consisting of: a tablet, a liquid dosage form, a hard gelatin capsule, a soft gelatin capsule, an HPMC capsule, an inhalant, an injectable, a transdermal, a buccal, a sublingual, and a rectal or a vaginal suppository; and where said compound is administered by a route selected from the group consisting of: peroral, intranasal, inhalation, parenteral, transdermal, rectal, vaginal, buccal, sublingual, or combination thereof.

26. The compound of claims 19, 20 and 23, where said one or more other substances is one or more of the following: pharmaceutically acceptable oil; lipid carrier; polymer; stabilizing agent; diluent; adjuvant; emulsifier; preservative; colorant; flavor imparting agent; acid; Tris base; sodium carbonate, or a combination thereof.

27. The compound of claim 23, where the compound contains not less than 1% by mass of one or more substances that exhibit in subjects at least partial activation of delta-opioid receptors.
